(19) United States
(12) Patent Application Publication
(10) Pub. No.: US 2010/0222543 A1
(43) Pub. Date: Sep. 2, 2010

(54) Process for the production of slow crystallizing polyester resin

(75) Inventors: Vikas Madhusudan Nadkarni, Maharashtra (IN); Srinivasacharya Ramacharya Ayodhya, Maharashtra (IN); Shreeram Ashok Wadekar, Maharashtra (IN); Ashwin Kumar Jain, Maharashtra (IN); Sachin Narayan Rane, Maharashtra (IN); Chetan Vijay Limaye, Maharashtra (IN)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Reliance Industries Ltd, Patalganga (IN)

(21) Appl. No.: 11/990,663

(22) PCT Filed: Aug. 14, 2006
(86) PCT No.: PCT/IN2006/000296

§ 371 Date: Feb. 19, 2008

(30) Foreign Application Priority Data
Aug. 22, 2005 (IN) 988/MUM/2005

(51) Int. Cl. C08G 63/16; C08G 63/183; C08G 63/189; C07C 69/82; C07C 69/76; C07C 63/14; C07C 31/18

(52) U.S. Cl. 528/308.3; 528/308; 560/76; 560/80; 562/480; 562/488; 568/852

(57) ABSTRACT

Improved process for the continuous production of slow crystallizing polyester resin comprising esterification of at least one dicarboxylic acid or mono-esters thereof or di-esters thereof with at least one polyol, melt polymerizing the esterified mixture, preparing uniform crystalline hemispherical prepolymer by particle former process, adding at least one diol having tertiary or quaternary carbon atoms with pendant groups such as neopentyl glycol or 2-methyl-1,3-propanediol or any suitable diol at any stage during esterification or melt polymerization but before the particle former process; and solid state polymerizing the prepolymer to obtain high molecular weight slow crystallizing polyester. Slow crystallizing low molecular weight crystalline hemispherical prepolymer having IV of about 0.1 dl/g to about 0.45 and uniform hemispherical shape, uniform particle size, uniform crystallinity prepared by the above process and is used as precursor to prepare high molecular weight resin. Slow crystallizing high molecular weight polyester resin with an IV from about 0.5 dl/g to about 1.2 dl/g having reduced melting point prepared by the above process and is used for the production of thick walled transparent preforms, large containers, films or sheets with accepted clarity.

### FIELD OF THE INVENTION

This invention relates to an improved process for the production of polyester resin comprising at least one diol having tertiary or quaternary carbon atoms with pendant groups such as neopentyl glycol or 2-methyl-1,3-propanediol or any suitable diol with slow rate of crystallization.

This invention also relates to an improved process for the production of crystalline prepolymer comprising at least one diol having tertiary or quaternary carbon atoms with pendant groups such as neopentyl glycol or 2-methyl-1,3-propanediol or any suitable diol, which is used as a precursor for solid state polymerization.

This invention also relates to polyester resin comprising at least one diol having tertiary or quaternary carbon atoms with pendant groups such as neopentyl glycol or 2-methyl-1,3-propanediol or any suitable diol with reduced melting point and slow rate of crystallization produced by the above process.

This invention also relates to crystalline prepolymer comprising at least one diol having tertiary or quaternary carbon atoms with pendant groups such as neopentyl glycol or 2-methyl-1,3-propanediol or any suitable diol, which is used as a precursor for solid state polymerization.

This invention also relates to use of polyester resin for the production of thick walled and transparent polyester preforms, containers, films or sheets.

### BACKGROUND OF THE INVENTION

PET resins are well known for making films, fibers and packaged container applications. Generally, two major and distinct process steps are involved in the production of high molecular weight polyesters. These two steps include melt polymerization and solid-state polymerization (SSP).

In the conventional polymerization process for producing polyester having high intrinsic viscosity (IV), base prepolymer of IV of about 0.4 dl/g to about 0.65 dl/g is produced by melt polymerization process. These base chips are cylindrical or spherical in shape. Base chip of prepolymer polyester is amorphous in nature. Base prepolymer is then subjected to solid-state polymerization after crystallizing it in a crystallizer so as to avoid sintering or lump formation in the SSP reactor. Using SSP process, depending on the application, different IV resin can be produced. Conventional polyester polymerization processes are disclosed in U.S. Pat. No. 3,405,098; U.S. Pat. No. 3,544,525; U.S. Pat. No. 4,245,253; U.S. Pat. No. 4,238,593; and U.S. Pat. No. 5,408,035.

Another polymerization process, which is different than conventional polymerization process, is disclosed in U.S. Pat. No. 5,510,454; U.S. Pat. No. 5,532,333; U.S. Pat. No. 5,540,868; U.S. Pat. No. 5,714,262; U.S. Pat. No. 5,830,982; and U.S. Pat. No. 6,451,966, which are incorporated herein as reference in their entirety. In this polymerization process, a crystalline hemispherical prepolymer having IV of about 0.1 dl/g to about 0.4 dl/g is formed using particle former process. This prepolymer is then be used as a precursor for solid state polymerization process to increase the IV from about 0.5 dl/g to about 1 dl/g. In this polymerization process, the residence time in the solid-state polymerizer reactor is 24 to 32 hours. Due to such longer residence time, the crystal perfection increases resulting in the higher melting temperature of the resin. This results in higher injection moulding temperatures. Higher processing temperature would result in higher energy consumption and hence higher cost of production. Also acetaldehyde content in the finished product such as preform and bottle would be on the higher side.

None of the processes reported in the prior art U.S. Pat. No. 5,510,454, U.S. Pat. No. 5,532,333, U.S. Pat. No. 5,540,868, U.S. Pat. No. 5,714,262, U.S. Pat. No. 5,830,982, and U.S. Pat. No. 6,451,966 disclose the polyester resin having neopentyl glycol as a co-monomer for reducing the crystal perfection for lower melting point.

Polyester resins are widely used as film for industrial uses and for food packaging. Polyester resins are also used in biaxially stretched bottles for packaging liquid foods and for producing large containers having volume more than five liters. Polyethylene terephthalate has excellent hygienic properties, impact resistance, heat resistance, transparency, gas barrier properties, chemical resistance, weatherability, etc. Polyethylene terephthalate (PET) resins are well known for the production of transparent containers, which are widely used in packaged water applications. The volume of such containers is usually in the range of about 0.25 liter to about 2 liter and the wall thickness is from about 0.2 mm to about 5 mm. These containers are produced by a stretch blow molding process in which injection moulded preforms having thickness of about 2 mm to about 10 mm are biaxially stretched to make a container of suitable shape. The clarity of preforms and bottles is essential for obtaining better market potential. If the rate of crystallization is very high, it imparts crystallinity in the preform thus making the preform hazy. If such crystalline preforms are blown to produce the bottles, the bottle will also loose the clarity and thus giving haze and further reducing the mechanical properties. In order to obtain fully amorphous preforms, the rate of crystallization of polyester must be very low. Hence, very low rate of crystallization of PET is an essential requirement for production of large thick walled transparent containers having volume from about 5 liters to about 30 liters. This is because; the thickness of the injection moulded preforms for such big containers is in the range from about 2 mm to about 10 mm and the resulting bottle thickness in the range from about 0.2 mm to 5 mm. Due to the higher thickness of preforms, in order to avoid crystallization, the cooling time for bringing the preform below the glass transition temperature increases. In order to avoid crystallization in the preform during cooling process, PET needs to be modified to exhibit very low crystallization rate.

In the prior art, PET was modified to minimize crystallization rate during parison molding and blow molding, by adding a small quantity of isophthalic acid (IPA) together with terephthalic acid as dicarboxylic acid component of PET, or cyclohexanedimethanol (CHDM) or neopentyl glycol together with ethylene glycol as glycol component of PET, thus producing copolymeric PET having a slow crystallizing rate by conventional copolymerization (Lecture abstracts of the 11^{th} Colloquium on Structure and Physical Properties of High Polymers, held by the Japanese High Polymer Society, Kanto Branch, on Jun. 16, 1981, “Recent Progress in Modification of Polymers” p. 3). These co-monomers are preferably added at the stage of polymerization

Polymerization process disclosed in U.S. Pat. No. 5,510,454; U.S. Pat. No. 5,532,333; U.S. Pat. No. 5,540,868; U.S. Pat. No. 5,714,262; U.S. Pat. No. 5,830,982; and U.S. Pat. No. 6,451,966, which are incorporated herein as reference in their entirety. In this polymerization process, a crystalline hemispherical prepolymer having IV of about 0.1 dl/g to about 0.4 dl/g is formed using the particle former process. This prepolymer can then be used as a precursor for solid-state polymerization process to increase the IV from about 0.5 dl/g to about 1 dl/g. Prepolymer quality can be judged by its onset of melting temperature, crystallinity, dust generation capability and particle shape and size distribution. These properties are associated with the crystallization behavior of prepolymer. Using this process, prepolymer having any additive or co-monomer that reduces the rate of crystallization, is extremely difficult since above mentioned properties deteriorate if rate of crystallization is lower.

Typically slow crystallizing polyester resin can be prepared by adding higher amount of IPA content. IPA content in the prepolymer plays vital role in solid state polymerization since high IPA content substantially reduces the crystal perfection of the prepolymer which further reduces onset melting temperature of the prepolymer. These factors compel solid state polymerization step to be carried out at lower temperature thereby reducing the productivity.

In order to have a consistent solid-state polymerization (SSP) process, quality of low IV prepolymer has to be good. Here the term “quality” means the uniformity in the crystalline morphology developed in the prepolymer during the particle former process. If the crystalline morphology of low IV prepolymer is non-uniform, it causes uneven solid-state polymerization reaction rate, lump formation, and high dust generation during the SSP process. In Good Manufacturing process low IV prepolymer has to be well crystallized on the particle former to avoid lump and dust formation during solid-state polymerization process.

### OBJECTS OF THE INVENTION

An object of the invention is to provide an improved process for the continuous production of low molecular weight crystalline polyester prepolymer having IV of 0.1 dl/g to 0.45 dl/g comprising at least one diol having tertiary or quaternary carbon atoms with pendant groups such as neopentyl glycol or 2-methyl-1,3-propanediol or any suitable diol along with conventional monomers where the prepolymer have uniform hemispherical shape, uniform particle size and uniform crystallinity with no or minimal reduction in its melting point.

Another object of the invention is to provide an improved process for the continuous production of high molecular weight crystalline polyester resin having IV of about 0.5 dl/g to about 1.2 dl/g from the low molecular weight crystalline prepolymer having IV of 0.1 dl/g to 0.45 dl/g comprising at least one diol having tertiary or quaternary carbon atoms with pendant groups such as neopentyl glycol or 2-methyl-1,3-propanediol or any suitable diol along with conventional monomers where the polyester resin have slow rate of crystallization and reduced melting point.

Yet another object of the invention is to provide low molecular weight crystalline polyester pre-polymer having IV of 0.1 dl/g to 0.45 dl/g comprising at least one diol having tertiary or quaternary carbon atoms with pendant groups such as neopentyl glycol or 2-methyl-1,3-propanediol or any suitable diol along with conventional monomers where the polyester prepolymer have uniform hemispherical shape, uniform particle size and uniform crystallinity with no or minimal reduction in its melting point.

Yet another object of the invention is to provide high molecular weight polyesters having IV of about 0.5 dl/g to about 1.2 dl/g comprising at least one diol having tertiary or quaternary carbon atoms with pendant groups such as neopentyl glycol or 2-methyl-1,3-propanediol or any suitable diol along with conventional monomers where the polyester resin have slow rate of crystallization and reduced melting point.

Yet another object of the invention is to provide use of high molecular weight polyesters having IV of about 0.5 dl/g to about 1.2 dl/g comprising at least one diol having tertiary or quaternary carbon atoms with pendant groups such as neopentyl glycol or 2-methyl-1,3-propanediol or any suitable diol along with conventional monomers for the production of thick walled transparent applications such as performs, container, films or sheets.

### DETAILED DESCRIPTION OF THE INVENTION

According to the invention there is provided an improved process for the continuous production of slow crystallizing high molecular weight polyester resin having IV of about 0.5 dl/g to about 1.2 dl/g from low molecular weight crystalline prepolymer having IV of about 0.1 dl/g to about 0.45 dl/g using particle former process, comprising at least one diol having tertiary or quaternary carbon atoms with pendant groups such as neopentyl glycol or 2-methyl-1,3-propanediol or any suitable diol along with conventional monomers.

According to the present invention, there is provided an improved process for the production of uniform hemispherical polyester prepolymer comprising at least one dicarboxylic acid or mono-esters or di-esters thereof, at least one polyol (The term “polyol” means alcohol having at least two or more hydroxyl group) and at least one diol having tertiary or quaternary carbon atoms with pendant groups such as neopentyl glycol or 2-methyl-1,3-propanediol or any suitable diol. The IV of the prepolymer is in the range of about 0.1 dl/g to about 0.45 dl/g. The base polyester chips produced with this process are crystalline in nature. These crystalline prepolymer chips further used as a precursor for solid-state polymerization for increasing the IV.

According to the invention there is provide an improved process for the continuous production of slow crystallizing polyester resin the process comprising:
a. esterifying among other things, at least one dicarboxylic acid selected from terephthalic acid, isophthalic acid, naphthalene dicarboxylic acid or 4,4′-biphenyl dicarboxylic acid or mono-esters thereof or di-esters thereof and at least one polyol selected from monoethylene glycol, diethylene glycol, triethylene glycol, propylene glycol, dipropylene glycol, butylenes glycol or 1,4-cyclohexane diol at temperature in the range of 250 to 290° C. and removing excess or unreacted polyol or water produced in the esterification to obtain an esterified mixture;
b. melt polymerizing the esterified mixture at temperature in the range of 260 to 300° C. to obtain low molecular weight polyester prepolymer having IV of about 0.1 to about 0.45 dl/g;
c. producing a crystalline and a hemispherical shape prepolymer by using particle former process at temperature in the range of 110 to 160° C.;
d. adding at least one diol having tertiary or quaternary carbon atoms with pendant groups such as neopentyl glycol or 2-methyl-1,3-propanediol or any suitable diol at any stage during esterification or melt polymerization but before the particle former process;
e. polymerizing a crystalline and hemispherical prepolymer by solid-state polymerization at temperature in the range of 200 to 240° C. to produce high molecular weight polyester resin with slow crystallizing property suitable for the production of thick walled and transparent preforms, containers, films or sheets.

The diol having tertiary or quaternary carbon atoms with pendant groups such as neopentyl glycol or 2-methyl-1,3-propanediol or any suitable diol is added in the range of 0.01% to 10% by weight of polyester. Preferably, the diol having tertiary or quaternary carbon atoms with pendant groups such as neopentyl glycol or 2-methyl-1,3-propanediol or any suitable diol is added in the range of about 0.5% to about 5% by weight of polyester. The diol having tertiary or quaternary carbon atoms with pendant groups such as neopentyl glycol or 2-methyl-1,3-propanediol or any suitable diol is added in the esterification reactor, oligomer line or column reactor particularly in the oligomer line but before the particle former process to produce polyester with slow rate of crystallization. The diol having tertiary or quaternary carbon atoms with pendant groups such as neopentyl glycol or 2-methyl-1,3-propanediol or any suitable diol is added to the resin at any stage of esterification or melt polymerization intended for reducing the rate of crystallization required for the production of thick walled performs, large containers, films or sheets with accepted clarity.

Anhydrides such as pyromellitic dianhydride or trimellitic anhydride can also be used instead of dicarboxylic acid for producing slow crystallizing resin. The high molecular weight polyester resin comprises any suitable additives for the improvement of any performance of polyester article.

Here weight % means the weight of the co-monomer with respect to the polyester.

The process for the continuous production of high molecular weight polyester resin having IV of about 0.5 dl/g to about 1 dl/g produced from low molecular weight crystalline prepolymer having IV of about 0.1 dl/g to about 0.4 dl/g using particle former process which is disclosed in U.S. Pat. No. 5,510,454 and incorporated herein as a reference in its entirety.

Solid state polymerization process for the production of high molecular weight PET from the low molecular weight crystalline prepolymer is disclosed in U.S. Pat. No. 5,510,454; U.S. Pat. No. 5,532,333; U.S. Pat. No. 5,540,868; U.S. Pat. No. 5,714,262; U.S. Pat. No. 5,830,982; and U.S. Pat. No. 6,451,966, which is incorporated herein as a reference in its entirety. However, none of these patents disclose the composition for the production of polyester resin having neopentyl glycol for reducing its rate of crystallization.

The term “polyester” used herein is intended to include polymer and copolymer of polyethylene terephthalate (PET) or any other polyester.

U.S. Pat. No. 4,415,727 discloses polyester resin comprising 2-methyl-1,3-propanediol produced by the conventional polymerization process for the production of thick-walled clear polyester bottles. While none of the process reported in the U.S. Pat. No. 5,510,454, U.S. Pat. No. 5,532,333, U.S. Pat. No. 5,540,868, U.S. Pat. No. 5,714,262, U.S. Pat. No. 5,830,982, and U.S. Pat. No. 6,451,966 disclosed polyester resin comprising 2-methyl-1,3-propanediol for slow crystallizing resin for large container applications.

According to the invention there is provided slow crystallizing low molecular weight crystalline hemispherical prepolymer having IV of about 0.1 dl/g to about 0.45 and having uniform hemispherical shape, uniform particle size, uniform crystallinity prepared by the above mentioned process.

According to the invention there is provided slow crystallizing low molecular weight crystalline hemispherical prepolymer comprises among other things, at least one dicarboxylic acid selected from terephthalic acid, isophthalic acid, naphthalene dicarboxylic acid or 4,4′-biphenyl dicarboxylic acid or mono-esters thereof or di-esters thereof and at least one polyol selected from monoethylene glycol, diethylene glycol, triethylene glycol, propylene glycol, dipropylene glycol, butylenes glycol or 1,4-cyclohexane diol and at least one diol having tertiary or quaternary carbon atoms with pendant groups such as neopentyl glycol or 2-methyl-1,3-propanediol or any suitable diol in the range of 0.01% to 10% by weight of polyester. The base prepolymer polyester chips produced with this process are crystalline in nature. These crystalline prepolymer chips further used as a precursor for solid-state polymerization for increasing the IV.

According to the invention there is provided slow crystallizing low molecular weight crystalline hemispherical polyethylene terephthalate (PET) prepolymer having IV of 0.1 dl/g to 0.45 dl/g comprising at least one diol having tertiary or quaternary carbon atoms with pendant groups such as neopentyl glycol or 2-methyl-1,3-propanediol or any suitable diol possessing lower rate of crystallization.

According to the invention there is provided slow crystallizing high molecular weight polyester resin with an IV from about 0.5 dl/g to about 1.2 dl/g having reduced melting point prepared by the above mentioned process.

According to the invention there is provided slow crystallizing high molecular weight polyester resin having IV about 0.5 dl/g to about 1.2 dl/g produced from low molecular weight crystalline prepolymer having IV of about 0.1 dl/g to about 0.45 dl/g, comprising among other things, at least one dicarboxylic acid selected from terephthalic acid, isophthalic acid, naphthalene dicarboxylic acid or 4,4′-biphenyl dicarboxylic acid or mono-esters thereof or di-esters thereof and at least one polyol selected from monoethylene glycol, diethylene glycol, triethylene glycol, propylene glycol, dipropylene glycol, butylenes glycol or 1,4-cyclohexane diol and at least one diol having tertiary or quaternary carbon atoms with pendant groups such as neopentyl glycol or 2-methyl-1,3-propanediol or any suitable diol in the range of 0.01% to 10% by weight of polyester.

According to the invention there is provided slow crystallizing high molecular weight polyethylene terephthalate (PET) having IV about 0.5 dl/g to about 1.2 dl/g produced from low molecular weight crystalline prepolymer having IV of about 0.1 dl/g to about 0.45 dl/g, comprising among other things, at least one diol having tertiary or quaternary carbon atoms with pendant groups such as neopentyl glycol or 2-methyl-1,3-propanediol or any suitable diol in the range of 0.01% to 10% by weight of polyester.

The slow crystallizing high molecular weight polyester resin also comprises any suitable additives for the improvement of any performance of polyester article. For example slow crystallizing high molecular weight polyester resin comprises slip additive for the improvement of slip performance of polyester article.

According to the present invention, there is provided slow crystallizing high molecular weight polyester resin prepared by the above process, which is used for the production of thick walled transparent preforms, containers, large containers like beverage, films or sheets with accepted clarity.

According to the invention, the process uses diol having tertiary or quaternary carbon atoms with pendant groups such as neopentyl glycol or 2-methyl-1,3-propanediol or any suitable diol to prepare slow crystallizing high molecular weight polyester with reduced melting point. The branched structure of diol disturbs the packing efficiency of the chains of polyester thereby reducing the rate of crystallization. However, addition of these co-monomers, diol having tertiary or quaternary carbon atoms with pendant groups such as neopentyl glycol or 2-methyl-1,3-propanediol or any suitable diol, does not reduces the crystal perfection to large extent thereby causing no or minimal reduction in the melting point of polyester prepolymer. According to the invention the Slow crystallizing low molecular weight crystalline hemispherical prepolymer obtained have uniform hemispherical shape, uniform particle size and uniform crystallinity with no or minimal reduction in its melting point. Further, the high molecular weight polyester resin obtained by the invention has slower crystallization rate and reduced melting point. Thus, the process gives a polyester with slower rate of crystallization, which is used in thick walled transparent applications such as container, preforms, sheets or films with acceptable clarity.

Although the invention has been described with reference to specific examples, it will be appreciated by those skilled in the art that the invention may be embodied in many other forms.

### Example 1

PET prepolymer of IV of 0.245 dl/g was prepared by melt-phase polymerization process. Purified terephthalic acid and monoethylene glycol (MEG) were charged in 1:2 ratio in reactor. 2 wt % Isophthalic acid and 1.5 wt % diethylene glycol (DEG) were added in the reactor. Esterification reaction was carried out at 280° C. About 1% wt neopentyl glycol was added in the oligomer line. The oligomer obtained was further polymerized at 290° C. to raise the IV up to 0.245 dl/g. About 290 ppm of antimony in the form of antimony trioxide was added as a catalyst and 10 ppm phosphorous in the form of phosphoric acid was added as thermal stabilizer. The low IV prepolymer melt was then passed through the 1.5 mm diameter orifice to form droplets on a continuous moving steel belt of particle former. These droplets were then crystallized on the particle former maintained at a temperature between 110 to 160° C. and then collected for carrying out solid-state polymerization. They were used as precursor for solid-state polymerization.

### Example 2

### Comparative

PET prepolymer of IV of 0.245 dl/g was prepared by melt-phase polymerization process. Purified terephthalic acid and monoethylene glycol (MEG) were charged in 1:2 ratio in reactor. 2 wt % Isophthalic acid and 1.5 wt % diethylene glycol (DEG) were added in the reactor. Esterification reaction was carried out at 280° C. The oligomer obtained was further polymerized at 290° C. to raise the IV up to 0.245 dl/g. About 290 ppm of antimony in the form of antimony trioxide was added as a catalyst and 10 ppm phosphorous in the form of phosphoric acid was added as a thermal stabilizer. The low IV prepolymer melt was then passed through the 1.5 mm diameter orifice to form droplets on a continuous moving steel belt of particle former. These droplets were then crystallized on the particle former maintained at a temperature between 110 to 160° C. and then collected for carrying out solid-state polymerization. The prepolymer obtained was used as precursor for solid-state polymerization. This prepolymer was considered as “Control”.

### Example 3

### Solid State Polymerization

Crystalline prepolymer having IV of 0.245 dl/g obtained in the example 1 to 2, was solid-state polymerized under inert atmosphere to raise the IV up to 1 dl/g. During SSP process, the material was passed through fluid bed heater at a temperature 236° C. with residence time of at least for 13 minutes. The material was further passed through crystallizer maintained at 224° C. and passed through a reactor of stage 1 maintaining temperature at 232° C. and gas to solid ratio 0.6 with at least two hours residence time. The material was further passed through a reactor of stage 2 maintaining temperature at 221° C. and gas to solid ratio 0.5 for at least 23 hours residence time.

The properties of hemispherical shaped low molecular crystalline prepolymer of PET with and without neopentyl glycol prepared according to Examples 1 and 2 are given in the table 1.

TABLE 1
Properties of hemispherical shaped low molecular crystalline
prepolymer of PET with and without neopentyl glycol

| | | | Carboxyl | | |
|---|---|---|---|---|---|
| | | Neopentyl | end | Tm | |
| | | glycol | groups | peak | Delta |
| Prepolymer | IV (dl/g) | (wt %) | (meq/kg) | (° C.) | Hm (J/g) |
| Prepolymer of | 0.245 | 0 | 141 | 256.0 | 48.0 |
| PET | | | | | |
| (prepared | | | | | |
| according to | | | | | |
| Example 2) | | | | | |
| Prepolymer of | 0.255 | 1 | 123 | 254.1 | 48.4 |
| PET | | | | | |
| (prepared | | | | | |
| according to | | | | | |
| Example 1) | | | | | |

Wherein Tm peak is the peak melting temperature and Delta Hm is the heat of fusion obtained using differential thermal calorimetry (DSC).

Results of table 1 indicated the similar melting temperatures and crystallinity in the prepolymer with and without neopentyl glycol. This is extremely important for trouble free run of SSP process.

Melting properties of high molecular weight resin with and without neopentyl glycol are given in the table 2.

TABLE 2
Melting properties of high IV resin with and without neopentyl
glycol

| | | | Melting temperatures | | | |
|---|---|---|---|---|---|---|
| | Neopetyl | | (° C.) | | | |
| | glycol | IV | Tm | Tm | Tm | DHm |
| Resin | (wt %) | (dl/g) | onset | peak | end | (J/g) |
| PET | 0 | 0.821 | 262.7 | 269.8 | 274.2 | 71.8 |
| (prepared according | | | | | | |
| to Examples 2 and 3) | | | | | | |
| PET | 1 | 0.870 | 256.0 | 263.7 | 269.7 | 65.4 |
| (prepared according | | | | | | |
| to Examples 1 and 3) | | | | | | |

Melting temperatures are obtained using DSC. Heating rate was 10° C./min.

In spite of higher IV of resin, addition of neopentyl glycol was found to reduce melting point to a larger extent.

The crystallization properties of high molecular weight resin with and without neopentyl glycol are given in the table 3.

TABLE 3
crystallization properties of high IV resin with and without
neopentyl glycol

| | | | | Spherulite size |
|---|---|---|---|---|
| | Neopentyl | | Tc onset | at 180° C. |
| Resin | glycol (wt %) | IV (dl/g) | ° C. (DSC) | (microns) |
| PET | 0 | 0.821 | 223.0 | 21.6 |
| (prepared | | | | |
| according to | | | | |
| Examples 2 | | | | |
| and 3) | | | | |
| PET | 1 | 0.870 | Not | 14.5 |
| (prepared | | | observed | |
| according to | | | | |
| Examples 1 | | | | |
| and 3) | | | | |

Wherein Tc onset is the onset of crystallization obtained by differential scanning calorimetry (DSC) during cooling from the melt state and the spherulite size obtained using hot-stage optical microscopy at 180° C. during cooling scan from the molten state.

Table 3 indicated that polyester containing neopentyl glycol did not crystallize as seen from the Tc onset temperature data obtained from DSC.

Optical microscopy was used to study the morphology development in the PET resin with and without neopentyl glycol. Spherulite size obtained with neopentyl glycol (prepared according to Examples 1 and 3) was much lower than that of Control indicating slower rate of crystallization.

Preforms having sidewall thickness of 9 mm was produced using each of the high IV polyester resin obtained in a comparative example 3 was moulded in a injection moulding machine. Preforms were blow moulded in a 20 L containers using blow-moulding machine. Table 4 indicates the injection and stretch blow moulding performance of the PET resin with or without neopentyl glycol (PET control).

TABLE 4
injection and stretch blow moulding performance of PET resin
with and without neopentyl glycol

| | | | Container | | |
|---|---|---|---|---|---|
| | | | color | | |
| | Neopentyl | Moulding | properties | | % Haze - |
| Resin | glycol (wt %) | temp (° C.) | L* | b* | Container |
| PET - | — | 270-280 | — | — | — |
| Control | | | | | |
| (prepared | | | | | |
| according to | | | | | |
| Examples 2 | | | | | |
| and 3) | | | | | |
| PET | 1 | 270-280 | 95.2 | 1.5 | 0.7 |
| (prepared | | | | | |
| according to | | | | | |
| Examples 1 | | | | | |
| and 3) | | | | | |

As given in the table 4, clear performs could not be obtained from PET control resin (Without neopentyl glycol). Preforms made with control resin were opaque thus not subjected to blow moulding process. Better clarity of performs as well as containers were observed with PET resin containing neopentyl glycol.

We claim:

1. An improved process for the continuous production of slow crystallizing polyester resin
the process comprising:
a. Esterifying among other things, at least one dicarboxylic acid selected from terephthalic acid, isophthalic acid, naphthalene dicarboxylic acid or 4,4′-biphenyl dicarboxylic acid or mono-esters thereof or di-esters thereof with at least one polyol selected from monoethylene glycol, diethylene glycol, triethylene glycol, propylene glycol, dipropylene glycol, butylenes glycol or 1,4-cyclohexane diol at a temperature in the range of 250 to 290° C. and removing excess or unreacted polyol or water produced in the esterification to obtain an esterified mixture;
b. melt polymerizing the esterified mixture at a temperature in the range of 260 to 300° C. to obtain low molecular weight polyester prepolymer having IV of about 0.1 to about 0.45 dl/g;
c. producing a crystalline and a hemispherical shape prepolymer by using particle former process at a temperature in the range of 110 to 160° C.;
d. adding at least one diol having tertiary or quaternary carbon atoms with pendant groups such as neopentyl glycol or 2-methyl-1,3-propanediol or any suitable diol at any stage during esterification or melt polymerization but before the particle former process; and
e. polymerizing the crystalline and hemispherical prepolymer by solid-state polymerization at a temperature in the range of 200 to 240° C. to produce high molecular weight polyester resin with slow crystallizing property suitable for the production of thick walled transparent preforms, containers, films or sheets.

2. Process as claimed in claim 1, wherein the diol having tertiary or quaternary carbon atoms with pendant groups such as neopentyl glycol or 2-methyl-1,3-propanediol or any suitable diol is added in the range of 0.01% to 10% by weight of polyester.

3. Slow crystallizing low molecular weight crystalline hemispherical prepolymer having IV of about 0.1 dl/g to about 0.45 dl/g and uniform hemispherical shape, uniform particle size, uniform crystallinity prepared by the process as claimed in claim 1.

4. Slow crystallizing prepolymer as claimed in claim 3, wherein the prepolymer comprises among other things, at least one dicarboxylic acid selected from terephthalic acid, isophthalic acid, naphthalene dicarboxylic acid or 4,4′-biphenyl dicarboxylic acid or mono-esters thereof or di-esters thereof; at least one polyol selected from monoethylene glycol, diethylene glycol, triethylene glycol, propylene glycol, dipropylene glycol, butylenes glycol or 1,4-cyclohexane diol and at least one diol having tertiary or quaternary carbon atoms with pendant groups such as neopentyl glycol or 2-methyl-1,3-propanediol or any suitable diol in the range of 0.01% to 10% by weight of polyester.

5. Slow crystallizing high molecular weight polyester resin with an IV from about 0.5 dl/g to about 1.2 dl/g having reduced melting point prepared by the process as claimed in claim 1.

6. Slow crystallizing high molecular weight polyester resin as claimed in claim 5, wherein the resin having IV about 0.5 dl/g to about 1.2 dl/g produced from low molecular weight crystalline prepolymer having IV of about 0.1 dl/g to about 0.45 dl/g, comprising among other things, at least one dicarboxylic acid selected from terephthalic acid, isophthalic acid, naphthalene dicarboxylic acid or 4,4′-biphenyl dicarboxylic acid or mono-esters thereof or di-esters thereof; at least one polyol selected from monoethylene glycol, diethylene glycol, triethylene glycol, propylene glycol, dipropylene glycol, butylenes glycol or 1,4-cyclohexane diol and at least one diol having tertiary or quaternary carbon atoms with pendant groups such as neopentyl glycol or 2-methyl-1,3-propanediol or any suitable diol in the range of 0.01% to 10% by weight of polyester.

7. Use of slow crystallizing high molecular weight polyester resin as claimed in claim 5 prepared by the process as claimed in claim 1, for the production of thick walled preforms or large containers, films or sheets with accepted clarity.
